(19) United States
(12) Patent Application Publication
(10) Pub. No.: US 2026/0098023 A1
(43) Pub. Date: Apr. 9, 2026

(54) METHOD FOR HYDROGENATION OF FURFURAL TO BIO-FUEL

(71) Applicant: KING FAHD UNIVERSITY OF PETROLEUM AND MINERALS, Dhahran (SA)

(72) Inventors: M. Nasiruzzaman SHAIKH, Dhahran (SA); Afnan Mohammed AJEEBI, Dhahran (SA); Huda Saeed ALGHAMDI, Khobar (SA); Zain Hassan Abdallah YAMANI, Dhahran (SA)

(73) Assignee: KING FAHD UNIVERSITY OF PETROLEUM AND MINERALS, Dhahran (SA)

(21) Appl. No.: 18/909,618

(22) Filed: Oct. 8, 2024

(51) Int. Cl. C07D 307/42

(52) U.S. Cl. CPC C07D 307/42

(57) ABSTRACT

A method of hydrogenation includes contacting a catalyst, including rhodium nanoparticles in an amount of 0.9 percent by weight (wt. %) to 1.1 wt. % based on the total weight of the catalyst supported on alumina, with furfural in the presence of ethanol to form a reaction mixture. The method further includes heating the reaction mixture at a pressure, at a temperature, and for a time, in a hydrogen gas atmosphere to form a hydrogenated product including furfuryl ethyl ether. At least 99 wt. % of the furfural is reacted to form the furfuryl ethyl ether and the furfuryl ethyl ether is formed with at least a 99 wt. % selectivity.

### STATEMENT OF ACKNOWLEDGEMENT

Support provided by the King Fahd University of Petroleum and Minerals (KFUPM) Consortium, King Fahd University of Petroleum and Minerals, Saudi Arabia, through Project H2FC2312 is gratefully acknowledged.

### BACKGROUND

### Technical Field

The present disclosure is directed towards the synthesis of biofuels from biomass-derived feedstocks, and more particularly, towards a method of hydrogenating furfural to biofuels using rhodium nanoparticles.

### Description of Related Art

The “background” description provided herein is for the purpose of generally presenting the context of the disclosure. The work of the presently named inventors, to the extent it is described in this background section, as well as aspects of the description that may not otherwise qualify as prior art at the time of filing, are neither expressly nor impliedly admitted as prior art against the present disclosure.

With the advancement of civilization, fossil-fuel demands have risen due to the increasing population and developing industries. In the past few decades, fossil fuel reserves have depleted at an alarming pace while world energy demands are at an all-time high. Alternative energy sources to fossil fuels are needed worldwide. To cater to the energy demands of the world, exploration and development of non-fossil fuel carbon energy sources are needed. Biomass conversion may be an efficient way to decrease global dependence on fossil fuels in the oil and gas industry [Kucherov, F. A. et al., Chemical transformations of biomass-derived C6-furanic platform chemicals for sustainable energy research, material science, and synthetic building blocks, ACS Sustain Chem Eng, 2018, 6, 7, 8064-8092]. Furfural is a bio-based platform molecule to be considered [Taylor, M. J. et al., Highly selective hydrogenation of furfural over supported Pt nanoparticles under mild conditions, Appl Catal B, 2016, 180, 580-585; and Chen, X. et al., Highly selective hydrogenation of furfural to furfuryl alcohol over Pt nanoparticles supported on G-C₃N₄ nanosheets catalysts in water, Sci Rep, 2016, 6, 1, 28558]. Furfural may be etherified to produce various fuel additives or surfactants through direct or reductive etherification pathways [Corma, A. and Renz, M. A., General method for the preparation of ethers using water-resistant solid Lewis acids, Angewandte Chemie International Edition, 2007, 46, 1-2, 298-300]. A reductive etherification process involves the acetalization of aldehydes as an intermediate, followed by hydrogenolysis and/or dehydration-hydrogenation of (semi) acetal [Wu, D. et al., In-situ generation of Brønsted acidity in the Pd-I bifunctional catalysts for selective reductive etherification of carbonyl compounds under mild conditions, ACS Catal, 2019, 9, 4, 2940-2948]. These processes found challenges in achieving a requisite ether yield as alcohol formation as a byproduct decreased the ether yield due to the absence of acid sites in the reductive etherification process. A bifunctional Pd/SiO₂—Al₂O₃-30 catalyst for selective hydrogenation-etherification of furfural achieves an approximate yield of 86.2%. Incorporating Si with Al₂O₃ in the catalyst reduced metal-support interactions, resulting in larger Pd particles and Brønsted acid centers. This strategy aims to produce effective bio-aldehydes for ether production [Yang, K. et al., Metal-acid dual sites in Pd/SiO₂—Al₂O₃ synergistically catalyze selective hydrogenation-etherification of furfural to bioether, J Catal, 2023, 425, 170-180]. Rh—ReOₓ/SiO₂, Rh—MoOₓ/SiO₂, and Rh—ReOₓ/C have been used as hydrogenation catalysts. Catalyst characterization indicated that rhodium (Rh) metal particles are directly modified with corresponding oxide species, indicating the interactions between metal oxide species and Rh metal surface may be a parameter for hydrogenation catalysis [Koso, S. et al., Comparative Study of Rh—MoOₓ and Rh—ReOₓ Supported on SiO₂ for the hydrogenolysis of ethers and polyols, Appl Catal B, 2012, 111-112, 27-37].

Although methods for the hydrogenation of furfural have been described in the past, there still exists a need to develop efficient methods for hydrogenation of furfural. An object of the present disclosure is to provide a method for hydrogenation of furfural to yield value-added products with increased selectivity and greater yields that may overcome drawbacks of the current art.

### SUMMARY

In an exemplary embodiment, a method of hydrogenation is described. The method includes contacting a catalyst, including rhodium nanoparticles supported on alumina, with furfural in the presence of ethanol to form a reaction mixture. The catalyst includes the rhodium nanoparticles in an amount of 0.9 percent by weight (wt. %) to 1.1 wt. % based on a total weight of the catalyst. The method further includes heating the reaction mixture at a pressure, at a temperature, and for a time, in a hydrogen gas atmosphere to form a hydrogenated product including furfuryl ethyl ether. At least 99 wt. % of the furfural is reacted to form the furfuryl ethyl ether, and the furfuryl ethyl ether is formed with at least a 99 wt. % selectivity.

In some embodiments, the catalyst includes rhodium nanoparticles in an amount of 1 wt. % based on a total weight of the catalyst.

In some embodiments, the catalyst includes rhodium nanoparticles in an amount of 0.1 wt. % to 5 wt. % based on a total weight of the catalyst.

In some embodiments, the method includes heating the reaction mixture in an autoclave.

In some embodiments, the reaction mixture is stirred at a speed of 200 revolutions per minute (rpm) to 300 rpm during the heating.

In some embodiments, the alumina is an alpha-alumina, α-Al₂O₃.

In some embodiments, the catalyst is in the shape of nanoparticles having an average particle size of 10 nanometers (nm) to 15 nm.

In some embodiments, the nanoparticles are agglomerated.

In some embodiments, the agglomerated nanoparticles form larger particles having a particle size of 5 micrometers (μm) to 20 μm separated by one or more crevices having a width of 1 μm to 5 μm and a length of 5 μm to 50 μm.

In some embodiment, the nanoparticles have a rhombohedral structure.

In some embodiments, the nanoparticles have a d-spacing of 0.15 nm to 0.3 nm.

In some embodiments, the method includes heating the reaction mixture at a pressure of 20 bar to 40 bar in a hydrogen atmosphere.

In some embodiments, the method includes heating the reaction mixture at a pressure of 25 bar to 35 bar in a hydrogen atmosphere.

In some embodiments, the method includes heating the reaction mixture at a temperature of 40 degrees Celsius (° C.) to 130° C.

In some embodiments, the method includes heating the reaction mixture at a temperature of 110° C. to 130° C.

In some embodiments, the method includes heating the reaction mixture for a time of 20 to 30 hours.

In some embodiments, the method includes heating the reaction mixture at for a time of 23 to 25 hours.

In some embodiments, the ethanol is neat ethanol.

In some embodiments, 0 wt. % of the furfural is reacted to form a difurfuryl ether.

In another exemplary embodiment, a method of making the catalyst is described. The method includes mixing rhodium nanoparticles with aluminum oxide for 10 minutes to 60 minutes to form a mixture. Further, the method includes heating the mixture at a temperature of 300° C. to 500° C. to form the catalyst.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a flowchart illustrating a method of hydrogenation, according to certain embodiments.

FIG. 1B is a flowchart illustrating a process for making a catalyst, according to certain embodiments.

FIG. 1C illustrates a schematic diagram of biomass conversion into biofuels, according to certain embodiments.

FIG. 2A illustrates a reaction sequence for reductive (direct) etherification of furfural (FF), according to certain embodiments.

FIG. 2B illustrates a reaction sequence for reductive (direct) etherification (top scheme), and hydrogenation and etherification (two-step) of furfural (FF) under alcohol, according to certain embodiments.

FIG. 2C illustrates a schematic diagram of a synthesis procedure of alumina support catalysts, according to certain embodiments.

FIG. 3A depicts a scanning electron microscopy (SEM) image of one weight percent rhodium on alumina support (1% Rh@Al₂O₃) at a scale of 10 micrometers (μm), according to certain embodiments.

FIG. 3B depicts an SEM image of 1% Rh@Al₂O₃ at a scale of 5 μm, according to certain embodiments.

FIG. 3C depicts an SEM image of elemental mapping of 1% Rh@Al₂O₃, according to certain embodiments.

FIG. 3D depicts an SEM image of elemental mapping of rhodium (Rh) in 1% Rh@Al₂O₃, according to certain embodiments.

FIG. 3E depicts a transmission electron microscopy (TEM) image of 1% Rh@Al₂O₃, at a scale of 50 nanometers (nm), according to certain embodiments.

FIG. 3F depicts a TEM image of 1% Rh@Al₂O₃, at a scale of 20 nm, according to certain embodiments.

FIG. 3G depicts a high-resolution transmission electron microscopy (HR-TEM) image of 1% Rh@Al₂O₃, at a scale of 10 nm, according to certain embodiments.

FIG. 3H depicts a selective area electron diffraction (SAED) image of 1% Rh@Al₂O₃, according to certain embodiments.

FIG. 4 is an X-ray diffraction (XRD) pattern of Al₂O₃ nanoparticles and 1% Rh@Al₂O₃, according to certain embodiments.

FIG. 5 is a graph summarizing catalytic test results of 1% Rh@Fe₃O₄, 1% Rh@Fe₂O₃, 1% Rh@Al₂O₃, 1% Rh@MgO, and 1% Rh@TiO₂, conducted at a temperature of 120 degrees Celsius (° C.) and a pressure of 30 bar in the presence of ethanol as solvent, according to certain embodiments.

FIG. 6A is a graph depicting the effect of reaction temperature on the hydrogenation of furfural using 1% Rh@Al₂O₃, according to certain embodiments.

FIG. 6B is a graph depicting the effect of rhodium loading percentage on the hydrogenation of furfural, according to certain embodiments.

### DETAILED DESCRIPTION

In the following description, it is understood that other embodiments may be utilized, and structural and operational changes may be made without departure from the scope of the present embodiments disclosed herein.

When describing the present disclosure, the terms used are to be construed in accordance with the following definitions, unless a context dictates otherwise. Embodiments of the present disclosure will now be described more fully hereinafter with reference to the accompanying drawings wherever applicable, in which some, but not all embodiments of the disclosure are shown. In the drawings, reference numerals designate identical or corresponding parts throughout the several views. Further, as used herein, the words “a,” “an,” and the like generally carry a meaning of “one or more,” unless stated otherwise. Within the description of this disclosure, where a numerical limit or range is stated, the endpoints are included unless stated otherwise. Also, all values and subranges within a numerical limit or range are specifically included as if explicitly written out.

Furthermore, the terms “approximately,” “approximate,” “about,” and similar terms generally refer to ranges that include the identified value within a margin of 20%, 10%, or preferably 5%, and any values therebetween.

Embodiments of the present disclosure will now be described more fully hereinafter with reference to the accompanying drawings, in which some, but not all embodiments of the disclosure are shown.

As used herein, the words “about,” “approximately,” or “substantially similar” may be used when describing magnitude and or position to indicate that the value and/or position described is within a reasonable expected range of values and/or positions. For example, a numeric value may have a value that is +/−0.1% of the stated value (or range of values), +/−1% of the slated value (or range of values), +/−2% of the stated value (or range of values), +/−5% of the slated value (or range of values), +/−10% of the staled value (or range of values), +/−15% of the stated value (or range of values), or +/−20% of the stated value (or range of values). Within the description of this disclosure, where a numerical limit or range is stated, the endpoints are included unless stated otherwise. Also, all values and subranges within a numerical limit or range are specifically included as if explicitly written out.

The present disclosure is intended to include all hydration states of a given compound or formula, unless otherwise noted or when heating a material.

In addition, the present disclosure is intended to include all isotopes of atoms occurring in the present compounds and complexes. Isotopes include those atoms having the same atomic number but different mass numbers. By way of general example, and without limitation, isotopes of hydrogen include deuterium and tritium.

As used herein, the term “catalyst” refers to a substance that speeds up a chemical reaction without being consumed in the process and remain unchanged after the reaction. Catalysts may react with one or more reactants to form intermediates that may subsequently give a reaction product and regenerate the catalyst in the process.

As used herein, the term “hydrogenation” refers to a chemical reaction between molecular hydrogen and another compound or element, usually in the presence of a catalyst. Hydrogenation may refer to a reduction reaction where hydrogen gas is added across multiple bonds of a molecule in the presence of a catalyst.

As used herein, “d-spacing” refers to the interplanar distance between adjacent parallel planes of atoms, ions, or molecules within a crystal lattice. This spacing is typically denoted as dₕₖₗ in the context of Miller indices, where h, k, and l are the Miller indices that define the orientation of the crystal planes.

Aspects of the present disclosure are directed to hydrogenation of furfural using a catalyst. The catalyst includes a varying weight percentage of rhodium nanoparticles supported on α-Al₂O₃ catalyst (support). The effect of the support, reaction conditions, and weight percentage of rhodium nanoparticles loaded on the support on the furfural hydrogenation indicate that 1% Rh@Al₂O₃ attained good conversion (about 99%) of furfural with good selectivity (about 99%) for ethyl furfuryl ether.

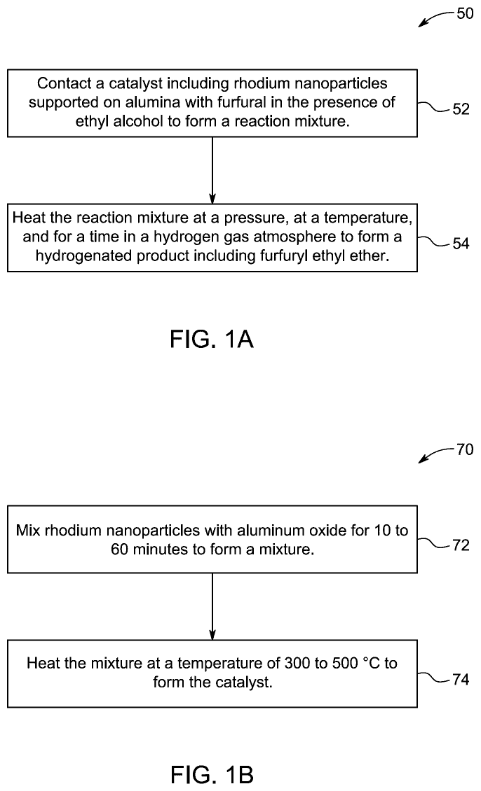

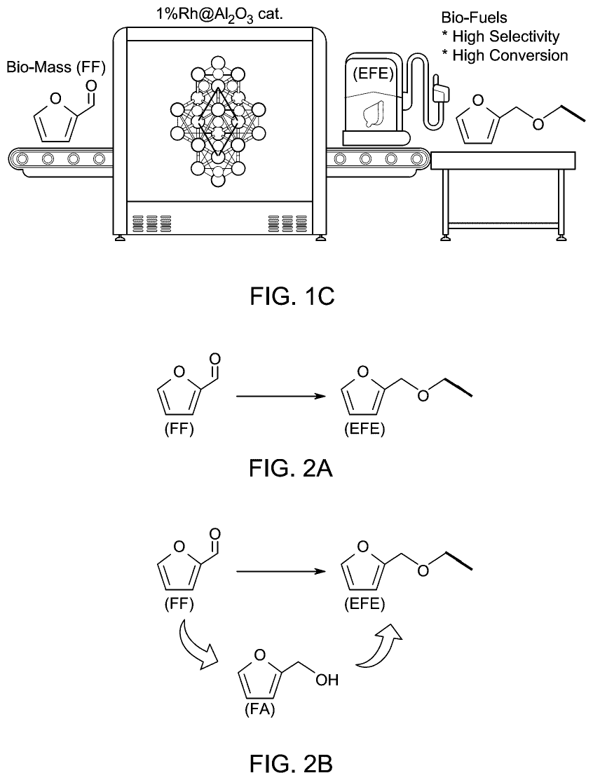

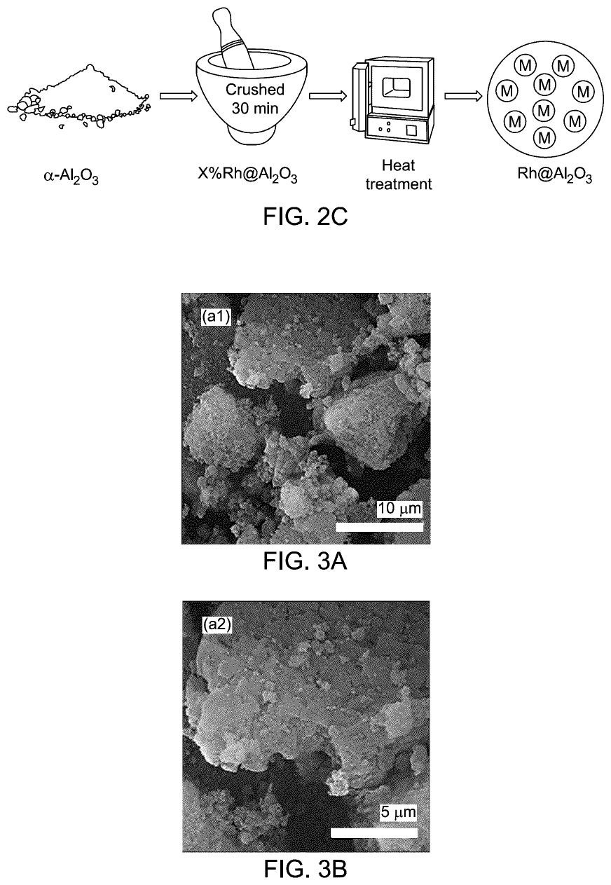

FIG. 1A illustrates a schematic flowchart of a method 50 of hydrogenation of furfural. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, method 50 includes contacting a catalyst, including rhodium nanoparticles supported on alumina, with furfural in the presence of ethanol, to form a reaction mixture. The catalyst includes rhodium nanoparticles in an amount of 0.9 to 1.1 wt. %, preferably 0.92 to 1.08 wt. %, preferably 0.94 to 1.06 wt. %, preferably 0.96 to 1.04 wt. %, preferably 0.98 to 1.02 wt. %, more preferably 0.99 to 1.01 wt. %, and yet more preferably about 1 wt. % based on the total weight of the catalyst. In a preferred embodiment, the catalyst includes rhodium nanoparticles in an amount of 1 wt. % based on the total weight of the catalyst. In other embodiment, the catalyst includes rhodium nanoparticles in an amount of 0.1 to 5 wt. %, preferably 0.5 to 3 wt. %, preferably 0.8 to 2 wt. %, and most preferably about 1 wt. % based on the total weight of the catalyst. The rhodium nanoparticles may be procured commercially or prepared from any of the rhodium salts by methods known in the art. In some embodiments, the alumina (aluminum oxide) may be present in any of the active or transition-form alumina, e.g., alpha-, gamma-, delta-, eta-, theta-, iota-, chi-, and kappa-alumina, mixtures thereof, and the like. In a preferred embodiment, the alumina is an alpha-alumina, α-Al₂O₃. Other supports, such as magnetite, hematite, magnesium oxide, and the like, may optionally be used in place of or in combination with aluminum oxide. In some embodiments, the alumina may further include oxides of silica, zirconia, lanthanum, titanium, carbon, mixtures thereof, and the like.

In some embodiments, the catalyst is in the shape of nanoparticles having an average particle size of 10 to 15 nm, preferably 11 to 14 nm, and preferably about 12 to 13 nm. The nanoparticles may exist in various morphological shapes, such as nanowires, nanocrystals, nanorectangles, nanotriangles, nanopentagons, nanohexagons, nanoprisms, nanodisks, nanocubes, nanoribbons, nanoblocks, nanobeads, nanotoroids, nanodiscs, nanobarrels, nanogranules, nanowhiskers, nanoflakes, nanofoils, nanopowders, nanoboxes, nanostars, tetrapods, nanobelts, nano-urchins, nanofloweres, mixtures thereof, and the like. In some embodiments, the nanoparticles are agglomerated. In some embodiments, the agglomerated nanoparticles are layered. In some embodiments, the agglomerated nanoparticles form larger particles having a particle size of 5 to 20 μm, preferably 6 to 18 μm, preferably 8 to 16 μm, preferably 10 to 15 μm, and preferably 12 to 14 μm, separated by one or more crevices having a width of 1 to 5 μm, preferably 2 to 4 μm, and a length of 5 to 50 μm, preferably 10 to 45 μm, preferably 15 to 40 μm, preferably 20 to 35 μm, and preferably 25 to 30 μm. In some embodiments, the one or more crevices are curved. In some embodiments, the crevices are branched. In some embodiments, a surface of the agglomerated nanoparticles is irregular with cracks having a length of 10 to 200 nm, preferably 20 to 150 nm, preferably 40 to 120 nm, and preferably 50 to 100 nm and a width or 0.1 to 5 nm, preferably 0.2 to 2.5 nm, and preferably 0.5 to 1 nm, and bumps having a diameter of 0.1 to 2 μm, preferably 0.2 to 1 μm, preferably 0.3 to 0.8 μm, and preferably 0.4 to 0.6 μm. In some embodiments, the cracks may be curved, branched, straight, and any other form known in the art. In some embodiments, the bumps may be raised features having a spherical form, a semi-spherical form, a rectangular form, and any other form known in the art. In some embodiments, the nanoparticles have a rhombohedral structure. In some embodiments, the nanoparticles have a d-spacing of 0.15 to 0.3 nm, preferably 0.16 to 0.28 nm, preferably 0.18 nm to 0.25 nm, more preferably 0.2 to 0.23 nm, and yet more preferably about 0.22 nm.

The catalyst is contacted with furfural. The weight ratio of furfural to the catalyst is in the range of 1:1 to 20:1, preferably 3:1 to 15:1, preferably 7:1 to 12:1, more preferably 9:1 to 11:1, and yet more preferably about 10:1. This reaction is carried out in the presence of alcohol, preferably ethanol. In some embodiments, the ethanol is neat ethanol. In some embodiments, the ethanol may be diluted with water or alcohol. Optionally, other alcohols, such as methyl alcohol, propanol, butanol, 1-pentanol, 2-pentanol, cyclohexanol, a combination thereof, and the like, may be used in combination with or in place of the ethanol.

At step 54, the method 50 includes heating the reaction mixture at a pressure, at a temperature, and for a time in a hydrogen gas atmosphere to form a hydrogenated product comprising furfuryl ethyl ether. The furfuryl ethyl ether is formed with at least a 99 wt. % selectivity. In some embodiments, the heating is carried out in an autoclave. In other embodiments, heating is carried out by any methods known in the art. In some embodiments, the reaction mixture is heated at a pressure of 20 to 40 bar, preferably 23 to 37 bar, preferably 25 to 35 bar, more preferably 28 to 32 bar, and yet more preferably at about 30 bar, in a hydrogen atmosphere. In some embodiments, the reaction mixture is heated to a temperature of 40 to 130° C., preferably 50 to 130° C., preferably 70 to 130° C., preferably 100 to 130° C., more preferably 110 to 130° C., and yet more preferably about 120° C. for 20 to 30 hours, preferably 22 to 26 hours, more preferably 23 to 25 hours, and yet more preferably for about 24 hours. In some embodiments, the reaction mixture is heated in an autoclave, although other heating appliances like ovens, microwaves, autoclaves, hot plates, heating mantles and tapes, oil baths, salt baths, sand baths, air baths, hot-tube furnaces, hot-air guns, combinations thereof, and the like may be used as well. In some embodiments, during the heating process, the reaction mixture is stirred at a speed of 200 to 300 revolutions per minute (rpm), preferably 220 to 280 rpm, more preferably 240 to 260 rpm, and yet more preferably about 250 rpm.

One factor affecting the percentage yield of furfuryl ether is the reaction temperature. The percentage yield may fluctuate with temperature change. In some embodiments, when the heating is carried out at about 120° C., at least 99 wt. % of the furfural is reacted to form the furfuryl ethyl ether, and less than 1%, preferably about 0% of the furfural is converted to a difurfuryl ether.

FIG. 1B illustrates a flow chart of a method 70 for making the catalyst. The order in which the method 70 is described is not intended to be construed as a limitation, and any number of the described method steps can be combined in any order to implement the method 70. Additionally, individual steps may be removed or skipped from the method 70 without departing from the spirit and scope of the present disclosure.

At step 72, the method 70 includes mixing rhodium nanoparticles with aluminum oxide for 10 to 60 minutes, preferably 20 to 40 minutes, more preferably 25 to 35 minutes, and yet more preferably about 30 minutes to form a mixture. In some embodiments, the aluminum oxide (alumina) may be present in any of the active or transition-form alumina, e.g., alpha-, gamma-, delta-, eta-, theta-, iota-, chi-, and kappa-alumina, mixtures thereof, and the like. In a preferred embodiment, the alumina is an alpha-alumina, α-Al₂O₃. Other supports, such as magnetite, hematite, magnesium oxide, and the like, may optionally be used in combination with or in place of the aluminum oxide. In some embodiments, the aluminum oxide may further include oxides of silica, zirconia, lanthanum, titanium, carbon, mixtures thereof, and the like. One factor that may affect the catalytic performance of the catalyst is the concentration of the rhodium nanoparticles loaded on the support. In an embodiment, the concentration of the rhodium nanoparticles is in the range of 0.1 to 5 wt. %, preferably 0.5 to 3 wt. %, preferably 0.9 to 1.1 wt. %, and yet more preferably about 1 wt. %. In some embodiments, the mixture may be ground for homogenization.

At step 74, the method 70 includes heating the mixture at a temperature of 300 to 500° C., preferably 330 to 470° C., preferably 350 to 450° C., more preferably 380 to 420° C., and yet more preferably about 400° C. to form the catalyst. In some embodiments, the heating may occur for 1 to 24 hours, preferably 2 to 20 hours, preferably 4 to 16 hours, preferably 6 to 12 hours, and preferably 8 to hours. The catalyst thus prepared may have a moisture content of less than 1%, preferably less than 0.5%, and preferably less than 0.2% after heating. In the present disclosure, the rhodium nanoparticles are impregnated on the support in a single step to form the catalyst for a time and cost-efficient process.

### EXAMPLES

The following examples demonstrate a method of hydrogenation using rhodium nanoparticles. The examples are provided solely for illustration and are not to be construed as limitations of the present disclosure, as many variations thereof are possible without departing from the spirit and scope of the present disclosure.

### Example 1: Catalyst Preparation

Rhodium nanoparticles of varying weight percentages (wt. %), including 0.5 wt. %, 1 wt. %, and 3 wt. % were impregnated on alumina support by physical mixing and grinding for 30 minutes and then treated with heat at 400° C. to form catalysts.

### Example 2: Hydrogenation of Furfural

In an autoclave, a reactor tube was filled with 10 milliliters (mL) of ethanol, 1 millimolar (mmol) of furfural, and 10 milligrams (mg) of catalyst. The reactor was repeatedly flushed with hydrogen gas (H₂) before being inflated to a pressure. Then, for 24 hours, a pressure autoclave was heated while being constantly stirring at 250 revolutions per minute (rpm). After, the reactor was depressurized and cooled to room temperature. The products, with the conversion and selectivity, were detected using gas chromatography-mass spectrometry (GC-MS), and a comparison to standards was made.

### Example 3: Characterization

Transmission electron microscopy (TEM) images were obtained using a JEOL JEM2100F transmission electron microscope. Data for X-ray diffraction (XRD) was collected using a Rigaku model Ultima-IV diffractometer and Cu-Kα radiation of a strength of 1.5405 angstrom (Å) at 40 kilovolts (kV) and 25 milliamperes (mA) over a 2θ range between 20° and 90°. Scanning electron microscopy (SEM) was performed using a JSM-6610LV scanning electron microscope. A Shimadzu 2010 GC-MS was used to identify catalytic products by matching the species to those in the Wiley Registry Mass Spectral Library, placing them based on their molecular ion (M⁺), and detecting mass fragmentation. Catalytic reactions were performed in Teflon-lined autoclaves from Autoclave Engineers, (Model #100 mL mini reactor, S/N; 043094E15-1), fitted with a pressure gauge and a mechanical stirrer.

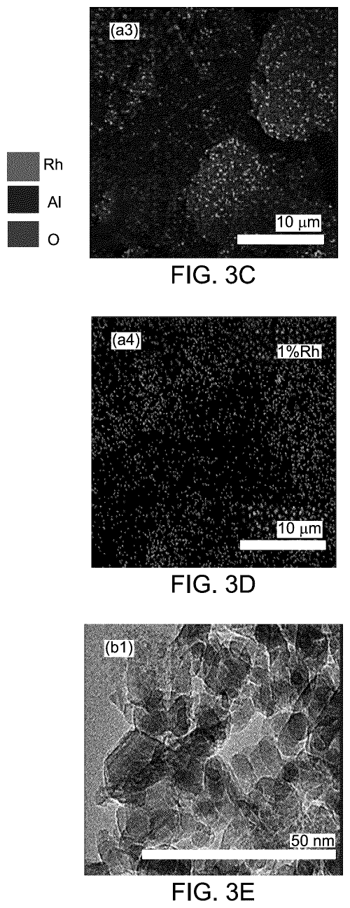

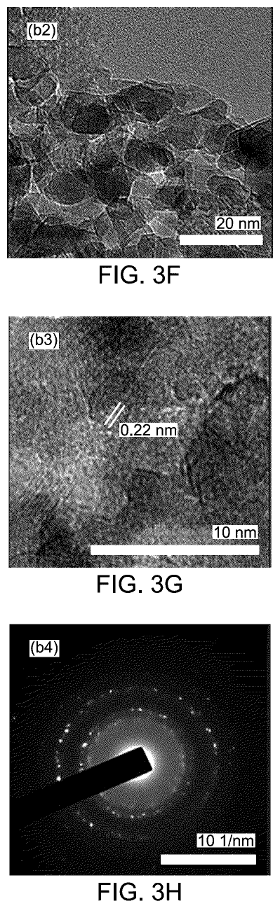

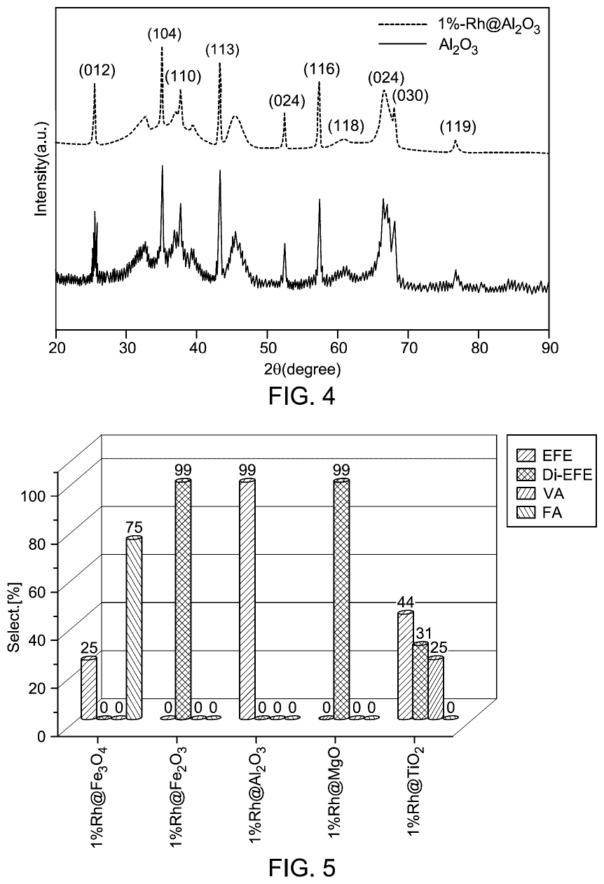

Morphological characteristics of the 1 wt. % rhodium (Rh) on alumina support (1% Rh@Al₂O₃) sample were described using SEM and TEM. As shown in FIGS. 3A-3H, optical images depict heterogeneous sizes, different shapes, spherical particles, and semi-spherical particles. The elemental map revealed a uniform and homogenous dispersion of aluminum and oxygen as support and rhodium (Rh) particles in the sample. The sample had a varied morphology and contained micron particle agglomerates with nanoparticles of different sizes. The average particle size of the sample examined by TEM was in the range of 11 nanometers (nm) to 12 nm. Optical images obtained by high-resolution transmission electron microscopy (HR-TEM) indicated the highly crystalline nature of the Al₂O₃ with a d-spacing value equal to 0.22 nm, as shown in FIG. 3G. XRD patterns of the prepared α-Al₂O₃ nanoparticles showed diffraction peaks of 20 to be around 25.4°, 35.0°, 37.7°, 43.8°, 57.4°, 68.1°, 52.5°, and 66.5° (FIG. 4). XRD patterns showed the nanoparticles have a rhombohedral and polycrystalline structure.

### Example 4: Effect of Catalyst Support on Hydrogenation of Furfural

The nature of oxide supports in catalysis is a factor that influences the catalytic activity and selectivity of a catalyst. Oxide supports can be employed in heterogeneous catalysis where the catalyst is a solid material and the reactants exist in either a gas or liquid phase. The oxide support acts as a platform or matrix on which catalytically active sites are either dispersed or anchored. The choice of an oxide support and a distribution of nanoparticles may have an impact on the side reactions between the furfural and the solvents. These surface properties can influence the interactions between the reactants and the active sites, thereby affecting catalytic reactions. For example, acidic supports may catalyze acid-catalyzed reactions, while basic supports may facilitate base-catalyzed reactions.

Performance of rhodium on various supports, such as 1 wt. % Rh on magnetite support (1% Rh@Fe₃O₄), 1 wt. % Rh on hematite support (1% Rh@Fe₂O₃), 1% Rh@Al₂O₃, 1 wt. % Rh on magnesium oxide support (1% Rh@MgO), and 1 wt. % Rh on titanium dioxide support (1% Rh@TiO₂) catalysts were investigated for the hydrogenation of furfural at a temperature of 120° C. and a pressure of 30 bar in the presence of ethanol as solvent, as seen in FIG. 5. The reaction between furfural and the alcohol solvent is desired for the reductive etherification pathway to form ethyl furfuryl ether (EFE). The order of the catalysts based on selectivity for EFE is as follows: 1% Rh@ Al₂O₃ (99%)>1% Rh@TiO₂ (44%)>1% Rh@Fe₃O₄ (25%)>1% Rh@MgO (0%) and 1% Rh@Fe₂O₃ (0%). The nature of the oxide support may regulate furfural decarboxylation toward selective hydrogenation. The acid and/or basic properties of the support may affect the etherification reaction in the hydrogenation of furfural. The base oxide catalysts, 1% Rh@MgO and 1% Rh@Fe₂O₃, have been found to hinder the etherification reactions and the hydrogenation reactions and resulted in 99% difurfuryl ether (Di-EFE). 1% Rh@Fe₃O₄ and 1% Rh@TiO₂ are neither acid nor base and did not show a selectivity to a reaction product. Acid-catalyzed reactions with 1% Rh@Al₂O₃ accomplished nearly full conversion with high selectivity (99%) for the EFE product compared to other catalysts at 120° C. and 30 bar.

### Example 5: Effect of Reaction Temperature on Hydrogenation of Furfural

The effect of reaction temperature on the hydrogenation of furfural is a factor that may impact reaction rate, selectivity, and overall outcome of the reaction process. Controlling the selectivity of the products may be challenging since the reaction pathway is influenced by the catalyst's structure and reaction conditions. Different reaction pathways may become dominant at various temperature ranges.

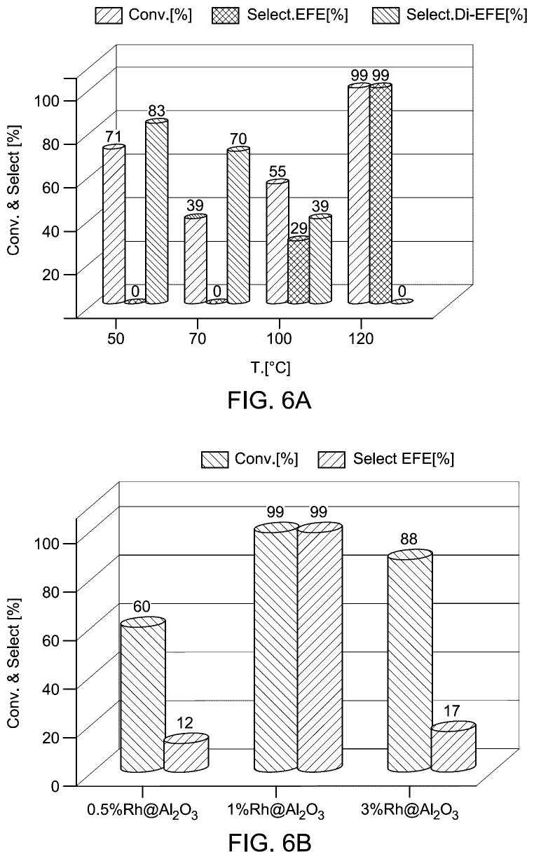

Experiments were performed at 50° C., 70° C., 100° C., and 120° C. to examine the effect of reaction temperature on product distribution. 1% Rh@Al₂O₃ was utilized in ethanol at a pressure of 30 bar H₂ for 24 hours, as seen in FIG. 6A. The results showed complete conversion (99%) and high selectivity (99%) for EFE at 120° C. and 30 bar. Reducing the temperature of the furfural (FF) hydrogenation process to 50° C. and 70° C. resulted in 83% and 70% selectivity for Di-EFE, respectively. Hydrogenation at 50° C. and 70° C. showed a 0% selectivity for EFE products, and additional undesired products were formed. Hydrogenation at 100° C. resulted in the percentage of Di-EFE formed being reduced to 39% and EFE increased to 29%. This indicates that higher temperatures may increase hydrogen solubility in the reaction medium, which may further promote the availability of hydrogen for a catalytic hydrogenation process.

### Example 6: Effect of Rhodium Loading Percentage on Hydrogenation of Furfural

Noble metal loading percentage may be an influential parameter in furfural hydrogenation. Loading percentages are considered to balance catalytic activity, selectivity, stability, and cost. Rhodium is an addition to the toolset for metal-catalyzed selective hydrogenations.

The concentration of noble metal loading on support affects catalytic performance. FIG. 6B illustrates the difference in selectivity of EFE production at 120° C. and 30 bar between 0.5% Rh@Al₂O₃, 1% Rh@Al₂O₃, and 3% Rh@Al₂O₃ to determine the influence of rhodium loadings on catalytic activity. 1% Rh@Al₂O₃ had the highest EFE selectivity value of 99%. This value is greater than 0.5% Rh@Al₂O₃ and 3% Rh@Al₂O₃, which provided 12% and 17% selectivity for EFE.

Hydrogenation of furfural for biofuel production, employing an acid catalyst such as 1% Rh@Al₂O₃, provides an efficient and environmentally friendly solution for sustainable energy generation. The method described in the present disclosure has experimentally demonstrated efficiency in converting furfural, a biomass-derived precursor, into valuable biofuels with high selectivity. 1% Rh@Al₂O₃ promotes selective chemical transformations and provides an advantage in terms of selectivity and conversion rates. 1% Rh@Al₂O₃ may propel the fuel industry towards a greener and more sustainable energy future.

Numerous modifications and variations of the present disclosure are possible in light of the above teachings. It is therefore to be understood that within the scope of the appended claims, the disclosure may be practiced otherwise than as specifically described herein.

1: A method of hydrogenation, comprising:
contacting a catalyst comprising rhodium nanoparticles supported on alumina with furfural in the presence of ethanol to form a reaction mixture,
wherein the catalyst comprises the rhodium nanoparticles in an amount of 0.9 to 1.1 wt. % based on a total weight of the catalyst,
heating the reaction mixture at a pressure, at a temperature, and for a time in a hydrogen gas atmosphere to form a hydrogenated product comprising furfuryl ethyl ether,
wherein at least 99 wt. % of the furfural is reacted to form the furfuryl ethyl ether,
wherein the furfuryl ethyl ether is formed with at least a 99 wt. % selectivity.

2: The method of claim 1, wherein the catalyst comprises rhodium nanoparticles in an amount of 1 wt. % based on a total weight of the catalyst.

3: The method of claim 1, wherein the catalyst comprises rhodium nanoparticles in an amount of 0.1 to 5 wt. % based on a total weight of the catalyst.

4: The method of claim 1, wherein the heating is carried out in an autoclave.

5: The method of claim 1, wherein the reaction mixture is stirred at a speed of 200 to 300 revolutions per minute (rpm) during the heating.

6: The method of claim 1, wherein the alumina is an alpha-alumina, α-Al₂O₃.

7: The method of claim 1, wherein the catalyst is in the shape of nanoparticles having an average particle size of 10 to 15 nm.

8: The method of claim 7, wherein the nanoparticles are agglomerated.

9: The method of claim 8, wherein the agglomerated nanoparticles form larger particles having a particle size of 5 to 20 μm separated by one or more crevices having a width of 1 to 5 μm and a length of 5 to 50 μm.

10: The method of claim 7, wherein the nanoparticles have a rhombohedral structure.

11: The method of claim 7, wherein the nanoparticles have a d-spacing of 0.15 to 0.3 nm.

12: The method of claim 1, wherein the heating occurs at a pressure of 20 to 40 bar in a hydrogen atmosphere.

13: The method of claim 1, wherein the heating occurs at a pressure of 25 to 35 bar in a hydrogen atmosphere.

14: The method of claim 1, wherein the heating occurs at a temperature of 40 to 130° C.

15: The method of claim 1, wherein the heating occurs at a temperature of 110 to 130° C.

16: The method of claim 1, wherein the heating occurs for a time of 20 to 30 hours.

17: The method of claim 1, wherein the heating occurs for a time of 23 to 25 hours.

18: The method of claim 1, wherein the ethanol is neat ethanol.

19: The method of claim 1, wherein 0 wt. % of the furfural is reacted to form a difurfuryl ether.

20: The method of claim 1, wherein the catalyst is made by a process comprising:
mixing rhodium nanoparticles with aluminum oxide for 10 to 60 minutes to form a mixture; and
heating the mixture at a temperature of 300 to 500° C. to form the catalyst.
